(19) United States
(12) Patent Application Publication
(10) Pub. No.: US 2014/0011830 A1
(43) Pub. Date: Jan. 9, 2014

(54) Artemisinin-Based Combination Therapy For Treating Parasitic Mediated Disease

(71) Applicant: Robert Lewis Steele, Palm Beach Gardens, FL (US)

(72) Inventor: Robert Lewis Steele, Palm Beach Gardens, FL (US)

(21) Appl. No.: 13/542,719

(22) Filed: Jul. 6, 2012

(51) Int. Cl. A61K 31/4375; A61P 33/06; A61P 33/00

(52) U.S. Cl. 514/280

(57) ABSTRACT

The present invention describes a method of treating individuals suffering from microbial infections, including a parasitic disease such as malaria, by using an improved Artemisinin Combination Therapy, known as Tri-ACT. The improved ACT therapy includes administering a combination of three drugs. In one embodiment of the present invention, the method includes administering to an individual a first composition comprising a therapeutically effective amount of an artemether spray sublingually. The individual is then administered a second composition, a therapeutically effective amount of artesunate. A third composition, an effective amount of berberine, or its pharmaceutically acceptable derivatives or salts is administered to the individual.

### REFERENCE TO RELATED APPLICATIONS

The present invention is related to U.S. patent application Ser. No. 13/542,702, entitled “ARTEMISININ-BASED COMBINATION THERAPY FOR TREATING VIRAL MEDIATED DISEASE” filed on Jul. 6, 2012. (Attorney Docket No. 4254U.001)

### FIELD OF THE INVENTION

The present invention relates to microbial therapies, and particularly to a method of treating individuals suffering from a parasitic related disease using an improved Artemisinin based therapy, and more particularly to a method of treating an individual suffering from malaria using an improved Artemisinin Combination Therapy (ACT).

### BACKGROUND OF THE INVENTION

Malaria is a serious and complex tropical parasitic disease spread by several species of mosquito. It is caused by a parasite from the Plasmodium genus, particularly Plasmodium falciparum, Plasmodium vivax, Plasmodium ovale, Plasmodium malariae, and Plasmodium knowlesi. The symptoms of malaria include fever, headaches, and vomiting usually after 10-15 days post mosquito bite. Left untreated, it may quickly develop into life-threatening complications. The Plasmodium parasites undergo complex life cycles once inside their human host. The life cycle starts with the mosquito bite of an infected female Anopheles mosquito. As a result of the bite, sporozoites are injected into the bloodstream, eventually reaching the liver. Once in the liver, the sporozoites multiply and release merozoites into the bloodstream where they invade the erythrocytes. In some infections, i.e. those caused by P. vivax, many parasites remain dormant in the liver. Those parasites in the liver can be reactivated, ultimately causing relapse. In addition to those remaining in the liver, many parasites can live and remain dormant in the gastrointestinal tract.

Malaria is a world wide problem with an estimated 500+ million cases in 2010. It is estimated that over 1.5 million people die every year from malaria, with most patients being children under the age of 5. A number of drugs ranging from natural drugs such as artemisinin and quinine, to synthetic drugs such as chloroquine, mefloquine, primaquine, halofantrine, amodiaquine, proguanil, and maloprim, have been developed to treat malaria. Despite the development of anti-malarial drugs, the number of infections and deaths related to the infection continue to rise. One factor resulting in the large number of malaria-related mortalities is the fact that Plasmodium falciparum, the deadliest malaria parasite, has acquired resistance against many available anti-malarial drugs. In fact, the World Health Organization (WHO) now prohibits the use of single compositions to fight the disease.

Therefore, what is needed in the art is an easy to administer method of treating individuals using an improved ACT methodology to individuals suffering from parasitic mediated disease, such as malaria.

### SUMMARY OF THE INVENTION

The present invention describes a therapy for individuals suffering from a parasitic infection based on Artemisinin Combination Therapy (ACT). In contrast to most ACT therapies which utilize a combination dual drug therapy, the present invention describes a method which uses a combination of three drugs. In one embodiment of the present invention, the method includes administering to an individual a first composition. The first composition comprises a therapeutically effective amount of an artemisinin derivate or its salt, such as an artemether spray delivered sublingually. The individual is then administered a second composition. The second composition comprises of a therapeutically effective amount of a second artemisinin derivate or its salt. The second artemisinin derivate differs from the first composition and is preferably artesunate. A third anti-microbial composition is then administered to the individual. The third composition comprises of an effective amount of berberine, or its pharmaceutically acceptable derivatives or salts. The second and third compositions are administered to the individual for additional periods, such as for two or three days.

In an alternative embodiment, the treatment is administered to an individual suffering from a disease transmitted by an arthropod, such as a mosquito or tick.

In an alternative embodiment, the treatment is administered to an individual suffering from a disease mediated by a eukaryotic protist of the genus Plasmodium.

In an alternative embodiment, the treatment is administered to a individual suffering from a disease mediated Plasmodium falciparum, Plasmodium vivax, Plasmodium ovale, Plasmodium malariae, and Plasmodium knowlesi.

In an alternative embodiment, the treatment is administered to an individual suffering from malaria.

In an alternative embodiment, the treatment is administered to an individual suffering from cerebral malaria.

As used herein, the term “pharmaceutically acceptable excipient” or “pharmaceutically acceptable carrier,” generally refers to organic or inorganic materials, non-toxic, inert solid, semi-solid or liquid filler, diluent, encapsulating material or formulation auxiliary of any type which cannot react with active ingredients. The excipients include but are not limited to sugars, such as lactose, glucose and sucrose; starches such as corn starch and potato starch; cellulose and its derivatives such as sodium carboxymethylcellulose, ethylcellulose, and cellulose acetate; powered tragacanth; malt; gelatin; talc; stearic acids; magnesium stearate; calcium sulfate; cocoa butter and suppository waxes; vegetable oils, such as peanut oil, cotton seed oil, seasame oil, olive oil, corn oil and oil of theobroma; esters such as but not limited to, ethyl oleate and ethyl laurate; polyols such as propylene glycol, glycerine, sorbitol, mannitol and polyethylene glycol; agar; alginic acids; buffering agents such as but not limited to, magnesium hydroxide and aluminum hydroxide; pyrogen-free water; isotonic saline; and phosphate buffer solution; skim milk powder; as well as other non-toxic compatible substances used in pharmaceutical formulations. Wetting agents and lubricants such as sodium lauryl sulfate and magnesium stearate, as well as coloring agents, flavoring agents, sweetening agents, lubricants, releasing agents, perfuming agents, carriers, tabletting agents, stabilizers, antioxidants and preservatives can also be present.

As use herein, “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “therapeutically effective amount” generally refers to an amount of an agent, for example the amount of a compound as an active ingredient, that is sufficient to effect treatment as defined herein when administered to an individual, such as a mammal, preferably a human in need of such treatment. A therapeutically effective amount of a compound, salt, analog, or derivative of the present invention will depend on a number of factors including, for example, the age and weight of the subject, the precise condition requiring treatment and its severity, the nature of the formulation, and the route of administration, and will ultimately be at the discretion of the attendant physician.

As used herein, the term “treat”, “treating” or “treatment” refers to the administration of therapy to a subject, particularly a mammal, more particularly a human, who already manifests at least one symptom of a disease to obtain a desired pharmacological and physiological effect. Such a subject includes an individual who is diagnosed as having a disease. The term may also include preventing the disease, i.e. causing the clinical symptoms of the disease not to develop in a mammal that may be exposed to or predisposed to the disease but does not yet experience or display symptoms of the disease, inhibiting the disease, i.e., arresting or reducing the development of the disease or its clinical symptoms, or relieving the disease, i.e., causing regression of the disease or its clinical symptoms.

Accordingly, it is an objective of the present invention to provide an improved Artemisinin Combination Therapy to individuals in need thereof.

It is a further objective of the present invention to provide an improved Artemisinin-based Combination Therapy to individuals suffering from a parasitic disease.

It is a further objective of the present invention to provide an improved Artemisinin-based Combination Therapy to individuals suffering from a parasitic disease transmitted by arthropods, such as a mosquito.

It is a still further objective of the present invention to provide an improved Artemisinin-based Combination Therapy to individuals suffering from a disease mediated by a eukaryotic protist of the genus Plasmodium.

It is a further objective of the present invention to provide an improved Artemisinin-based Combination Therapy incorporating a sublingual delivery step to individuals suffering from a parasitic disease.

It is yet another objective of the present invention to provide an improved Artemisinin-based Combination Therapy incorporating a sublingual delivery step to individuals suffering from a disease mediated by a eukaryotic protist of the genus Plasmodium.

It is a still further objective of the present to provide an improved Artemisinin-based Combination Therapy incorporating delivery of an artemisinin derivate using a sublingual, spray-based delivery route of administration to individuals suffering from malaria.

It is yet another objective of the present invention to provide an improved Artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from a parasitic disease.

It is a further objective of the present invention to provide an improved Artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from malaria.

It is yet another objective of the present invention to provide an improved Artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from cerebral malaria.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### DETAILED DESCRIPTION OF THE INVENTION

While the present invention is susceptible of embodiment in various forms, there is demonstrated and will hereinafter be described a presently preferred, albeit not limiting, embodiment with the understanding that the present disclosure is to be considered an exemplification of the present invention and is not intended to limit the invention to the specific embodiments illustrated.

The present invention describes a novel combination therapy for the treatment of microbial infections, such as parasitic infections. As an illustrative example, the present invention has been found to be useful in treating diseases mediated by arthropods and/or a plasmodium based parasites causing malaria. The combination therapy in accordance with the present invention is based on Artemisinin Combination Therapy (ACT). In accordance with the World Health Organization (WHO) regulations for the use of artemisinin based treatments, such compounds are generally combined with other drugs to minimize resistance. The present invention uses a tri-therapy modality, using a unique combination of three compositions: artemether, artesunate, and berberine. While most drugs currently used kill parasites in the blood stream, parasites in the gut remain where they or their eggs lodge and lay dormant for years. Such dormancy accounts for re-occurrence of the disease years later. Use of the tri-therapy allows for killing of the parasites in both the blood (artemesinin derivatives) and the digestive tract (berberine). In addition, the tri-therapy in accordance with the present invention allows for quick administration to patients suffering cerebral malaria. Administering the first composition by a sublingual spray not only allows the drug to enter the patient's body rapidly, but allows delivery to patients who may not be able to receive dosages via pills and eliminates any problem with using needles. Follow up doses of the second and third compositions ensure that the parasites are killed both in the blood and the digestive tract, thereby eliminating re-occurrence.

Artemisinin and Artemisinin Derivatives

Artemisinin, having an IUPAC name of 3R,5aS,6R,8aS,9R,12S,12aR)-octahydro-3,6,9-trimethyl-3,12-epoxy-12H-pyrano[4,3-j]-1,2-benzodioxepin-10(3H)-one and also known as Qinhaosu, is a natural product derived from the Chinese herb Artemisia annua known to have anti-viral activities, see Efferth et al. The Antiviral Activities of Artemisinin and Artesunate. Clin. Inf. Dis. 2008:47:804-11. It has a chemical structure of:

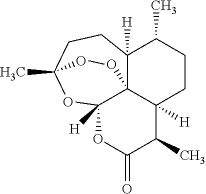 [SMILES: C[C@@H]1CC[C@H]2[C@@H](C)C(=O)O[C@@H]3O[C@]4(C)CC[C@@H]1[C@]32OO4]

Artemisinin is a sesquiterpene lactone containing an unusual peroxide bridge. This peroxide is believed to be responsible for the drug's mechanism of action. Few other natural compounds with such a peroxide bridge are known, see Artemisinin and a new generation of antimalarial drugs”. Education in Chemistry. July 2006. http://www.rsc.org/Education/EiC/issues/2006July/Artemisinin.asp. It is widely used for the treatment of malaria, particularly in combination with other drugs such as mefloquine (ASMQ), lumefantrine (such as sold under the trade name Coartem), amodiaquine (such as sold under the trade names Camoquin, Flavoquine, ASAQ), piperaquine (such as sold under the trade name Duo-Cotecxin), artemisinin and pyronaridine (such as sold under the trade name Pyramax). Artemisinin derivatives are typically used as prodrugs of the biologically active metabolite dihydroartemisinin, which is active during the stage when the parasite is located inside red blood cells. The mechanism through which artemisinin derivatives kill the parasite is believed to act via perturbing redox homeostasis in malaria parasites. Accordingly, such compositions may include free-radical production in the parasite food vacuole and inhibition of a parasite calcium ATPase. A key advantage of artemisinins is rapid action against all of the erythrocytic stages of the parasite, including transmissible gametocytes, resulting in a rapid clinical benefit and decreased transmission of malaria, see Rosenthal, Artesunate for Treatment of Severe Falciparum Malaria, N. Engl J Med 2008, 358:1829-1836.

One of the disadvantages of artemisinin is its poor physical properties resulting in poor bioavailability, limiting its effectiveness. For example, the compound is sparingly soluble in either water or oils and thus not readily absorbable by the gastrointestinal tract. As a result, numerous semi-synthetic derivatives have been developed, including artesunate (water-soluble: for oral, rectal, intramuscular, or intravenous use), artemether (lipid-soluble: for oral, rectal or intramuscular use) dihydroartemisinin, artelinic acid, artenimol and artemotil. Accordingly, the Artemisinin derivatives include but are not limited to artesunate, dihydroartemisinin, dihydroartemisinin hemisuccinate, dihydrodroartemisinin succinate, sodium artesunate, stabilized forms of artesunate, stabilized forms of sodium artesunate, dihydroartemisitene dimers as described in U.S. Pat. No. 7,098,242, amino-functionalized 1,2,4-trioxanes as described in U.S. Pat. No. 7,071,226, artemisinin endoperoxides as described in U.S. Pat. No. 6,984,640, spiro and dispiro 1,2,4-trioxolane anti-malarials as described in U.S. Pat. No. 6,906,205, mixed steroidal 1,2,4,5-tetraoxane compounds as described in U.S. Pat. No. 6,906,098, arteether as described in U.S. Pat. No. 6,750,356, substituted 1,2,4-trioxanes as described in U.S. Pat. No. 6,737,438, Artemisia annua extracts as described in U.S. Pat. No. 6,685,972, artemether as described in U.S. Pat. No. 6,683,193, trioxane derivatives based on artemisinin as described in U.S. Pat. No. 6,649,647, trioxane dimer compounds as described in U.S. RE38,117, conjugates of artelinic acid as described in U.S. Pat. No. 6,461,603, arteethers from dihydroartemisinin as described in U.S. Pat. No. 6,346,631, artemisinine or artemisinene derivatives as described in U.S. Pat. No. 6,306,896, C-10 carbon substituted artemisinin-like trioxane compounds as described in U.S. Pat. No. 6,160,004, water-soluble trioxanes as described in U.S. Pat. No. 6,136,847, alpha arteether as described in U.S. Pat. No. 6,127,405, artemisinin dimers as described in U.S. Pat. No. 5,856,351, (+)-deoxoarteminisinin and analogs of (+)-deoxoartemisinin as described in U.S. Pat. No. 5,225,562, and 10-substituted ether derivatives of dihydroartemisinin as described in U.S. Pat. No. 5,225,427, as well as its salts or other derivatives thereof as known to one of skill in the art.

Artemether is a methyl-ether derivative of dihydroartemisinin derived from artemisinin. It has an IUPAC chemical name of (+)-(3-alpha,5a-beta,6-beta,8a-beta, 9-alpha,12-beta,12aR)-decahydro-10-methoxy-3,6,9-trimethyl-3,12-epoxy-12H-pyrano(4,3-j)-1,2-benzodioxepin, having a chemical formula:

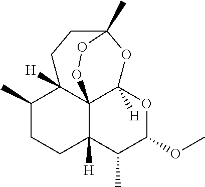

Artemether is known to be effective against blood schizots of several malaria causing parasites, and is used as an ACT drug.

Artesunate, also known as dihydroartemisinin hemisuccinate and its salts, has a IUPAUC chemical name of 3R,5aS,6R,8aS,9R,10S,12R,12aR)-Decahydro-3,6,9-trimethyl-3,12-epoxy-12H-pyrano(4,3-j)-1,2-benzodioxepin-10-ol hydrogen succinate, with a chemical formula of:

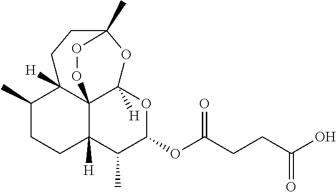

Artesunate is used primarily as treatment for malaria.

Dosage for Artemisinin, Artemisinin Salts and Derivatives.

The oral dosage range for artemisinin, artemisinin salts and derivatives is between about 1 mg to about 1,500 mg per dose administered one to three times daily. More preferably, the oral dosage range for Artemisinin, Artemisinin salts and derivatives is between about 20 mg to about 250 mg per dose if taken one to three times daily. Most preferably, the oral dosage ranges for artemisinin, artemisinin salts and derivatives is between about 40 mg to about 100 mg per dose taken one to three times daily.

Berberine, Salts Thereof, and Berberine Derivatives.

Berberine is a quaternary ammonium salt from the protoberberine group of isoquinoline alkaloids. It has a IUPAC name of (5,6-Dihydro-9,10-dimethoxybenzo[g]-1,3-benzodioxolo[5,6-a]quinolizinium), with a chemical formula of:

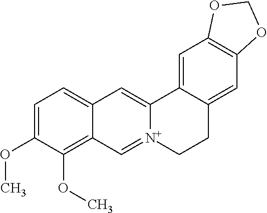 [SMILES: COc1ccc2cc3[n+](cc2c1OC)CCc1cc2c(cc1-3)OCO2]

It is found in various plant species of Berberis (e.g. Berberis aquifolium (Oregon grape), Berberis vulgaris (Barberry), and Berberis aristata (Tree Turmeric)), as well as other plant families, including but not limited to Hydrastis canadensis (Goldenseal), Phellodendron amurense (Amur Cork Tree, Huang Bai, Huang Po, Po Mu) and Coptis chinensis (Chinese Goldthread, Huang-Lian, Huang-Lien), and Tinospora cordifolia, and to a smaller extent in Argemone mexicana (Prickly Poppy) and Eschscholzia californica (Californian Poppy), Rhizoma coptidis Huanglian Jiedu decoction, San-Huang-Xie-Xin-Tang, Xietianwu, Gegen Quinlian, and Shizhu. Berberine is primarily isolated from the roots, rhizomes, stems, and bark. Berberine has been used for medical use in both Ayurvedic and Chinese medicines, and recently has been tested for antibacterial activity and use in diarrhea, prostate cancer and diabetes. It is generally considered to be non-toxic and shows no genotoxic activity.

In accordance with the present invention, berberine, berberine derivatives, or its salts may include, but not limited to, berberine alkaloid, berberine base, berberinehydrochloride, berberine, berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine, tetrahydroprotoberberine 8-cyanodihydroberberine, dimeric protoberberine alkaloids, demethylated protoberberine alkaloids, quataternary protoberberine alkaloids, protoberberineand protoberberine alkaloids, the salts of berberine, including berberine hydrochloride, berberine chloride, berberine sulfate, berberine tannate and other salts known to one of skill in the art.

Dosage Range for Berberine, its Salts and Derivatives.

The oral dosage range of berberine is from about 50 mg to about 1,500 mg administered in a single dose two to three times daily, not to exceed 4,500 mg per day. More preferably, the dose of berberine is about 100 mg to about 1,000 mg in a single human dose not to exceed 3,000 mg per day, taken one to three times daily. The most preferable dosage range for a single dose of berberine is about 200 to about 500 mg taken one to three times daily.

The present invention is further described by the following non limiting examples. The following examples illustrate embodiments, albeit preferred embodiments, of routes of administration and forms of the composition. While the preferred forms and/or routes are described, other forms, such as but not limited to tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration. The compositions may also be developed for inhalational, oral, rectal, vaginal, parenteral, topical, transdermal, pulmonary, intranasal, buccal, ophthalmic, intrathecal, intravenous or any other route of administration

The dosage form of the present invention may include either immediate or controlled release forms. Each composition can be formulated and manufactured by procedures known to one of skill in the art, and may include 100% composition or a mixture of one or more pharmaceutically acceptable excipients or pharmaceutically acceptable carriers. For use of sublingual sprays, liquid artemether can be prepared as a spray form using, for example, microfluidization process as described in U.S. Pat. No. 6,861,066.

### Example 1

### Method of Treating an Individual Suffering from a Disease Mediated by a Parasite

A method of treating an individual suffering from disease mediated by a parasite, preferably plasmodial parasites which cause malaria comprises the steps of (1) administering to a mammal, preferably a human, a first composition, the first composition comprising a therapeutically effective amount of an artemisinin derivative, preferably artemether at 50-80 mg per dose, preferably 60 mg (2) administering to the mammal a second composition, the second composition comprising a therapeutically effective amount of a second artemisinin derivate, the second artemisinin derivate differing from the first composition, and preferably being artesunate or its pharmaceutically acceptable derivatives at 1 mg-1500 mg per individual dose, preferably about 20 mg to about 250 mg per dose, administered three times daily, and most preferably about 40 mg to about 100 mg per dose administered three times daily; and (3) administering to a mammal a third composition, the third composition comprising a therapeutically effective amount of berberine, or its pharmaceutically acceptable derivatives at 1 mg to about 1500 mg per individual dose, particularly about 100 mg to about 1000 mg per dose three times a day, and more specifically about 200 mg to about 400 mg per dose, two to three times a day.

The following describes an illustrative, albeit preferred delivery treatment schedule:

Day 1: Delivery of artemether—single dose.
Delivery of artesunate and berberine tablets, dosage according to user age and/or weight every 12 hours.

Day 2: Delivery of artesunate and berberine
tablets, dosage according to user age and/or weight every 24 hours.

Day 3: Delivery of artesunate and berberine
tablets, dosage according to user age every 24 hours.

Day 4: Delivery of artesunate and berberine
tablets, dosage according to user age every 24 hours.

### Example 2

### Treatment for Adult Humans Over 165 Pounds (75 Kg) Having a Parasitic Infection Using Sublingual Delivery of an Artemether Solution: Malaria

A method of treating an individual suffering from malaria, including acute, chronic, or cerebral malaria, comprises the steps of (1) administering to an adult mammal, preferably a human, a first composition, the first composition comprising a therapeutically effective amount of artemether; (2) administering to the adult mammal a second composition, the second composition comprising a therapeutically effective amount of artesunate or its pharmaceutically acceptable derivatives; and (3) administering to the adult mammal a third composition, the third composition comprising a therapeutically effective amount of berberine, or its pharmaceutically acceptable derivatives.

In a preferred embodiment, the method of treatment can be facilitated by the use of a kit comprising a sublingual spray containing artemether solution as a single dosage unit spray bottle, a first packet containing artesunate tablets, and a second packet containing berberine tablets. An adult human, over 165 pounds (75 Kg) suffering from malaria, including acute, chronic, or cerebral malaria, begins the method in accordance with the present invention by administering the first loading dose of the artemether. The artemether is delivered to the individual using a sublingual spray bottle containing 60 mg of artemether solution per bottle. Use of a spray offers advantages over injectable form by providing a method of delivery that is easy to administer, does not require trained medical professionals such as a doctor or nurse, and avoids the problems associated with needles, such as fear of spreading disease such as HIV or Hepatitis C and proper disposal. In addition, sublingual drug delivery, particularly sprays, allows the active ingredients improved absorption and enhanced bioavailability.

In use, the user simply removes the bottle from the transportation/delivery packaging and displaces any safety features, such as any tamper proofing outside wrapping, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes the user administers the second composition comprising a dosage of artesunate, such as 3×50 mg tablets for the first 12 hours, followed by a repeat dosage the next 12 hours (300 mg per day). The user then takes 2 tablets every 24 hours (100 mg) for 3 additional days. The user administers the third composition comprising a dosage of berberine, such as 2×400 mg tablets for the first 12 hours followed by a repeat dosage the next 12 hours (1600 mg per day). The user then takes 2 tablets every 24 hours (800 mg) for 3 additional days. Alternatively, the second and third compositions can be administered simultaneously with the first composition.

### Example 3

### Treatment for Adult Human 66 (30 Kg)-165 Pounds (75 Kg) Suffering a Parasitic Infection Using Sublingual Delivery of an Artemether Solution: Malaria

In a preferred embodiment, the method of treatment can be facilitated by the use of a kit comprising a sublingual spray containing artemether solution as a single dosage unit spray bottle, a first packet containing artesunate tablets, and a second packet containing berberine tablets. An adult human 66 (30 kg)-165 pounds (75 Kg) suffering from malaria, including acute, chronic, or cerebral malaria, begins the method in accordance with the present invention by administering the first loading dose of artemether. The artemether is delivered to the individual using a sublingual spray bottle containing 60 mg artemether solution per bottle. The user simply removes the bottle from the transportation/delivery packaging and displaces any safety features, such as outside wrapping to indicate tampering, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes, the user administers the second composition comprising a dosage of artesunate, such as 2×50 mg tablets for the first 12 hours, followed by a repeat dosage the next 12 hours (200 mg per day). The user then takes 1 tablet every 24 hours (50 mg) for 3 additional days. The user administers the third composition comprising a dosage of berberine, such as 1×400 mg tablets for the first 12 hours, followed by a repeat dosage the next 12 hours (800 mg per day). The user then takes 1 tablet every 24 hours (400 mg) for 3 additional days. Alternatively, the second and third compositions can be administered simultaneously with the first composition.

### Example 4

### Treatment for Human Child 33 Pounds (15 kg)-66 Pounds (30 Kg) Suffering a Parasitic Infection Using Sublingual Delivery of an Artemether Solution: Malaria

For children or individual in the range of between 33 pounds (15 kg)-66 pounds (30 Kg) suffering from malaria, including acute, chronic, or cerebral malaria, the treatment is the same as described in Example 3.

### Example 5

### Treatment for Infants and Toddlers 33 Pounds Up to 3 Years Old (Under 15 Kg) Suffering a Parasitic Infection Using Sublingual Delivery of an Artemether Solution: Malaria

For infants and toddlers 33 pounds up to 3 years old (under 15 kg) suffering from malaria, including acute, chronic, or cerebral malaria, the treatment is similar to Example 4. The dosage delivered to the user includes artemether delivered using a sublingual spray bottle containing 60 mg artemether solution per bottle, 25 mg artesunate per dosage, 1 dose every 12 hours for the first day (50 mg total per day) followed by 1 dose (25 mg) every day for three days, and 1×200 mg berberine dose every 12 hours for the first day (400 mg total per day) followed by 1 dose (200 mg) every day for three days. For infants and toddlers, the tablets can be ground up and mixed with liquids that allow delivery to the user, such as milk, juice, and syrups.

### Example 6

### Treatment for Individuals Suffering a Parasitic Infection Using Injectable Delivery of Artemether Solution an Artemether Solution: Malaria

A method of treating an individual suffering from malaria comprises the steps of (1) administering to a mammal, preferably a human, a first composition, the first composition comprising a therapeutically effective amount of artemether; (2) administering to the mammal a second composition, the second composition comprising a therapeutically effective amount of a artesunate or its pharmaceutically acceptable derivatives; an (3) administering to the mammal a third composition, the third composition comprising a therapeutically effective amount of berberine, or its pharmaceutically acceptable derivatives.

In an illustrated embodiment, the method of treatment can be facilitated by the use of a kit comprising an injectable artemether solution as a single dosage unit contained in an ampoule, injection vial, or as an individual single, preloaded injection syringe with needle, a first packet containing artesunate tablets, and a second packet containing berberine tablets. An individual begins the method in accordance with the present invention by administering the first loading dose of the artemether. The artemether is delivered to the individual as an injection, via IV or IM route of administration. The second and third compositions are delivered as previously described in Examples 2-5, depending on the weight and age of the individual user.

As described in the above examples, the components of the present invention can be supplied in a kit to aid in delivery and use in areas where distribution channels and/or medical communities are not easily accessible or established. In accordance with the present invention, the kit may include a secure delivery package in the form of a self sealing blister pack, zip lock packs, standup pouches, foil pouches which include the single use spray bottle and one day, two day, three day, four day, five plus day packs of the artesunate and berberine. If injectable artemether is used, needles, syringes, injection bottles, or pre-loaded syringes with capped needles may be included. The kit preferably contains directions and/or any other instructions for the proper use and storage of the components of the kit.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of treating an individual suffering from a eukaryotic protist mediated disease transmitted by an arthropod comprising the steps of:
administering to an individual a first composition, said first composition comprising a therapeutically effective amount of an artemisinin derivate;
administering to said individual a second composition, said second composition comprising a therapeutically effective amount of a second artemisinin derivate, said second artemisinin derivate differing from said first composition;
administering to said individual a third composition comprising an effective amount of berberine, or its pharmaceutically acceptable derivatives.

2. The method of treating an individual suffering from a eukaryotic protist mediated disease transmitted by an arthropod according to claim 1 wherein said arthropod is a mosquito.

3. The method of treating an individual suffering from a eukaryotic protist mediated disease transmitted by an arthropod according to claim 1 wherein at least one of said compositions result in elimination of said protist within in the blood of said user.

4. The method of treating an individual suffering from a eukaryotic protist mediated disease transmitted by an arthropod according to claim 1 wherein at least one of said compositions result in elimination of said protist located in the digestive tract of said individual.

5. The method of treating an individual suffering from a eukaryotic protist mediated disease transmitted by an arthropod according to claim 1 wherein said first composition is administered sublingually.

6. The method of treating an individual suffering from a eukaryotic protist mediated disease transmitted by an arthropod according to claim 5 wherein said sublingual delivery is via a spray.

7. The method of treating an individual suffering from a eukaryotic protist mediated disease transmitted by an arthropod according to claim 1 wherein said first composition is administered to said individual via an injection.

8. The method of treating an individual suffering from a eukaryotic protist mediated disease transmitted by an arthropod according to claim 1 wherein said second and third compositions are delivered via an oral route.

9. The method of treating an individual suffering from a eukaryotic protist mediated disease transmitted by an arthropod according to claim 8 wherein second and third compositions are in the form of a pill, tablet, or capsule.

10. The method of treating an individual suffering from a eukaryotic protist mediated disease transmitted by an arthropod according to claim 8 further including the steps of administering additional doses of said second and said third compositions to said individual for a predetermined time period.

11. The method of treating an individual suffering from a eukaryotic protist mediated disease transmitted by an arthropod according to claim 1 wherein said individual has malaria.

12. The method of treating an individual suffering from a eukaryotic protist mediated disease transmitted by an arthropod according to claim 1 wherein said individual has cerebral malaria.

13. A method of treating an individual suffering from malaria comprising the steps of:
administering to an individual a first composition, said first composition comprising a therapeutically effective amount of artemether or its pharmaceutically acceptable salt;
administering to said individual a second composition, said second composition comprising a therapeutically effective amount of artesunate or its pharmaceutically acceptable salt;
administering to an individual a third composition, said third composition comprising a therapeutically effective amount of berberine, or its pharmaceutically acceptable salt.

14. The method of treating an individual suffering from malaria according to claim 13 wherein said first composition is administered sublingually.

15. The method of treating an individual suffering from malaria according to claim 14 wherein said sublingual delivery is via a spray.

16. The method of treating an individual suffering from malaria according to claim 14 wherein said first composition is administered to said individual via an injection.

17. The method of treating an individual suffering from malaria according to claim 13 wherein said second and said third compositions are delivered via an oral route.

18. The method of treating an individual suffering from malaria according to claim 17 wherein said second and said third compositions are in the form of a pill, tablet, or capsule.

19. The method of treating an individual suffering from malaria according to claim 18 wherein said step of administering said artemether includes delivering a single dosage to said individual.

20. The method of treating an individual suffering from malaria according to claim 13 further including the steps of administering additional doses of said second and said third compositions for a predetermined time period.

21. The method of treating an individual suffering from malaria according to claim 13 wherein said second and said third compositions are delivered in a solid form.
